(19) United States
(12) Patent Application Publication
(10) Pub. No.: US 2009/0306052 A1
(43) Pub. Date: Dec. 10, 2009

(54) INDENYL DERIVATIVES AND USE THEREOF FOR THE TREATMENT OF NEUROLOGICAL DISORDERS

(75) Inventor: David Matthew Wilson, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited

(21) Appl. No.: 11/720,950

(22) PCT Filed: Dec. 5, 2005
(86) PCT No.: PCT/EP05/13070

§ 371 Date: Jul. 9, 2009

(30) Foreign Application Priority Data
Dec. 7, 2004 (GB) 0426828.0
Sep. 19, 2005 (GB) 0519089.7

(51) Int. Cl. A61K 31/55; A61K 31/497; A61K 31/4439; C07D 403/12; C07D 401/12; C07D 403/14

(52) U.S. Cl. 514/217.04; 514/255.05; 514/343; 540/597; 544/408; 546/276.4

(57) ABSTRACT

The present invention relates to novel indenyl derivatives having pharmacological activity, processes for their preparation, to compositions containing them and to their use in the treatment of neurological and psychiatric disorders.

The present invention relates to novel indenyl derivatives having pharmacological activity, processes for their preparation, to compositions containing them and to their use in the treatment of neurological and psychiatric disorders.

WO2004/080968 (Eli Lilly and Company) describes a series of 6-substituted nicotinamide derivatives. The compounds are stated to be opioid receptor antagonists and are claimed to be useful in the treatment of obesity. WO01/03680 (Isis Innovation Ltd) discloses a series of compounds disclosed to be useful for inhibiting IAPP-associated amyloidosis. WO2004/052370 (7TM Pharma A/S) discloses a series of quinilone compounds that are stated to be useful in the treatment of disorders including obesity. WO02/098363 (Agouron Pharmaceuticals, Inc.) discloses a series of compounds capable of inhibiting the effect of gonadotropin-releasing hormone. GB2292558 describes a series of compounds capable of inhibiting the binding of fibrinogen to the platelet membrane. EP1188747 describes phenoxypropylamine compounds that are agonists of the 5-HT_{1A} receptor and are stated to be of use as antidepressants. WO2004/034963, WO03/092606, WO03/024456, WO01/66114 and EP0742207 (Eisai Co. Ltd.) disclose a series of cholinesterase inhibitors for the treatment of a number of diseases including Alzheimer's disease, dementia, migraine and injuries caused by organophosphorus compounds. WO05/00131 (Cambridge Neuroscience Incorporated) describes a series of piperidine derivatives and their use in the treatment of CNS disorders. U.S. Pat. No. 4,745,110 (Rorer Pharmaceutical Corporation), U.S. Pat. No. 4,647,559 (William H. Rorer, Inc.) and WO8404247 (Rorer International (Overseas) Inc.) describe a series of bicyclic benzenoid aminoalkylene ethers and thioethers and the use of these compounds in the treatment of gastrointestinal hypersensitivity and ulcerogenic disorders. U.S. Pat. No. 39,343,149 and U.S. Pat. No. 3,906,032 (E.R. Squibb & Sons, Inc.) describe a series of compounds for use as hypocholesteremic agents and anti-inflammatory agents.

The histamine H3 receptor is predominantly expressed in the mammalian central nervous system (CNS), with minimal expression in peripheral tissues except on some sympathetic nerves (Leurs et al., (1998), Trends Pharmacol. Sci. 19, 177-183). Activation of H3 receptors by selective agonists or histamine results in the inhibition of neurotransmitter release from a variety of different nerve populations, including histaminergic and cholinergic neurons (Schlicker et al., (1994), Fundam. Clin. Pharmacol. 8, 128-137). Additionally, in vitro and in vivo studies have shown that H3 antagonists can facilitate neurotransmitter release in brain areas such as the cerebral cortex and hippocampus, relevant to cognition (Onodera et al., (1998), In: The Histamine H3 receptor, ed Leurs and Timmerman, pp 255-267, Elsevier Science B.V.). Moreover, a number of reports in the literature have demonstrated the cognitive enhancing properties of H3 antagonists (e.g. thioperamide, clobenpropit, ciproxifan and GT-2331) in rodent models including the five choice task, object recognition, elevated plus maze, acquisition of novel task and passive avoidance (Giovanni et al., (1999), Behav. Brain Res. 104, 147-155). These data suggest that novel H3 antagonists and/or inverse agonists such as the current series could be useful for the treatment of cognitive impairments in neurological diseases such as Alzheimer's disease and related neurodegenerative disorders.

The present invention provides, in a first aspect, a compound of formula (I) or a pharmaceutically acceptable salt thereof:

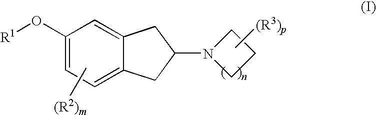

wherein:
R¹ represents —C₃₋₆ alkyl, —X—C₃₋₈ cycloalkyl, —X-aryl, —X-heterocyclyl, —X-heteroaryl, —X—C₃₋₈ cycloalkyl-Y—C₃₋₈ cycloalkyl, —X—C₃₋₈ cycloalkyl-Y-aryl, —X—C₃₋₈ cycloalkyl-Y-heteroaryl, —X—C₃₋₈ cycloalkyl-Y-heterocyclyl, —X-aryl-Y—C₃₋₈ cycloalkyl, —X-aryl-Y-aryl, —X-aryl-Y-heteroaryl, —X-aryl-Y-heterocyclyl, —X-heteroaryl-Y—C₃₋₈ cycloalkyl, —X-heteroaryl-Y-aryl, —X-heteroaryl-Y-heteroaryl, —X-heteroaryl-Y-heterocyclyl, —X-heterocyclyl-Y—C₃₋₈ cycloalkyl, —X-heterocyclyl-Y-aryl, —X-heterocyclyl-Y-heteroaryl, —X-heterocyclyl-Y-heterocyclyl;
X represents a bond or C₁₋₆ alkyl;
Y represents a bond, C₁₋₆ alkyl, CO, CONH, COC₂₋₆ alkenyl, O, SO₂ or NHCOC₁₋₆ alkyl;
R² represents halogen, C₁₋₆ alkyl, C₁₋₆ alkoxy, cyano, amino or;
m represents an integer from 0 to 2;
n represents an integer from 1 to 4;
R³ represents hydrogen, fluorine or —C₁₋₃ alkyl;
p represents an integer from 0 to 2;
wherein said alkyl, cycloalkyl, aryl, heteroaryl and heterocyclyl groups of R¹ may be optionally substituted by one or more substituents (e.g. 1, 2 or 3) which may be the same or different, and which are selected from the group consisting of halogen, hydroxy, cyano, nitro, ═O, haloC₁₋₆ alkyl, halo C₁₋₆ alkoxy, C₁₋₆ alkyl, C₁₋₆ alkoxy, aryl C₁₋₆ alkoxy, C₁₋₆ alkylthio, C₁₋₆ alkoxy C₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁴, —CO₂R⁴, —COR⁴, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵CO₂R⁶, —NR⁴CONR⁵R⁶ or —SO₂NR⁵R⁶ (wherein R⁴, R⁵ and R⁶ independently represent hydrogen, C₁₋₆ alkyl, —C₃₋₈ cycloalkyl, —C₁₋₆ alkyl-C₃₋₈ cycloalkyl, aryl, heterocyclyl or heteroaryl or —NR⁵R⁶ may represent a nitrogen containing heterocyclyl group, wherein said R⁴, R⁵ and R⁶ groups may be optionally substituted by one or more substituents (e.g. 1, 2 or 3) which may be the same or different, and which are selected from the group consisting of halogen, hydroxy, C₁₋₆ alkyl, C₁₋₆ alkoxy, cyano, amino, ═O or haloC₁₋₆ alkyl) provided that where R¹ represents —C₃₋₆ alkyl, R¹ is not substituted by hydroxy, C₁₋₆ alkoxycarbonyl or —CO₂R⁴;
or solvates thereof.

In one aspect, the alkyl, cycloalkyl, aryl, heteroaryl and heterocyclyl groups of R¹ may be optionally substituted by one or more substituents (e.g. 1, 2 or 3) which may be the same or different, and which are selected from the group consisting of halogen, hydroxy, cyano, nitro, ═O, sulfonyl, —R⁴, —CO₂R⁴, —COR⁴, or a group —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵CO₂R⁶, —NR⁴CONR⁵R⁶ or —SO₂NR⁵R⁶, —C₁₋₆ alkyl-SONHR⁷, —OSO₂R⁷, —C₁₋₆ alkyl-CONHR⁷, —C₁₋₆ alkyl-SO₂R⁷, —SO₂R⁷, —OR⁸, —O—C₁₋₆ alkyl-R⁹, —CO—C₁₋₆ alkylR⁹, —C₁₋₆ alkyl-COR⁹, —SOR¹⁰, —C₁₋₆ alkyl-OR¹⁰, —S—R¹⁰ (wherein R⁴, R⁵ and R⁶ independently represent hydrogen, —C₁₋₆ alkyl, —C₃₋₈ cycloalkyl, —C₁₋₆ alkyl-C₃₋₈ cycloalkyl, aryl, heterocyclyl or heteroaryl, wherein R⁷ represents —C₁₋₆ alkyl or aryl, wherein R⁸ represents —C₁₋₆ alkyl, —C₁₋₆ alkyl-C₃₋₈ cycloalkyl or aryl, wherein R⁹ represents aryl and wherein R¹⁰ represents —C₁₋₆ alkyl, and wherein —NR⁵R⁶ may represent a nitrogen containing heterocyclyl group, and wherein said R⁴, R⁵ and R⁶ groups may be optionally substituted by one or more substituents (e.g. 1, 2 or 3) which may be the same or different, and which are selected from the group consisting of halogen, hydroxy, C₁₋₆ alkyl, C₁₋₆ alkoxy, cyano, amino, ═O or haloC₁₋₆ alkyl) provided that where R¹ represents —C₃₋₆ alkyl, R¹ is not substituted by hydroxyl or —CO₂R⁴.

In another aspect, R¹ represents —X—C₃₋₈ cycloalkyl, —X-aryl, —X-heterocyclyl, —X-heteroaryl, —X—C₃₋₈ cycloalkyl-Y—C₃₋₈ cycloalkyl, —X—C₃₋₈ cycloalkyl-Y-aryl, —X—C₃₋₈ cycloalkyl-Y-heteroaryl, —X—C₃₋₈ cycloalkyl-Y-heterocyclyl, —X-aryl-Y—C₃₋₈ cycloalkyl, —X-aryl-Y-aryl, —X-aryl-Y-heteroaryl, —X-aryl-Y-heterocyclyl, —X-heteroaryl-Y—C₃₋₈ cycloalkyl, —X-heteroaryl-Y-aryl, —X-heteroaryl-Y-heteroaryl, —X-heteroaryl-Y-heterocyclyl, —X-heterocyclyl-Y—C₃₋₈ cycloalkyl, —X-heterocyclyl-Y-aryl, —X-heterocyclyl-Y-heteroaryl or —X-heterocyclyl-Y-heterocyclyl,

In a further aspect in which R¹ represents —X-heteroaryl, —X-heteroaryl-Y-heterocyclyl or X-heteroaryl-Y—C₃₋₈ cycloalkyl, wherein X represents C₁₋₆alkyl and Y represents a bond, the heteroaryl group is other than a quinolinyl group.

In yet another aspect in which R¹ represents —X-heteroaryl, —X-heteroaryl-Y-aryl, —X-heteroaryl-Y-heteroaryl, —X-heteroaryl-Y-heterocyclyl or X-heteroaryl-Y—C₃₋₈ cycloalkyl, wherein X represents a bond and Y represents CONH or SO₂, the heteroaryl group is other than a furanyl group.

In a further aspect in which R¹ represents —X-heterocyclyl-Y-aryl or X-heterocyclyl-Y-heteroaryl wherein the heterocyclyl group is a piperidinyl, piperizinyl or a dihydro-2H-pyridin-1-yl group, the X group is other than an optionally substituted C₃alkyl group

In another aspect in which R¹ represents —X-heteroaryl or —X-heteroaryl-Y-aryl wherein X represents C₁₋₆alkyl and Y represents a bond or C₁₋₆alkyl, the heteroaryl group is other than a tetrazolyl group.

In one aspect in which R¹ represents —X—C₃₋₈ cycloalkyl or —X-aryl wherein X represents C₁₋₆alkyl, the X group is not substituted by a C₁₋₆ alkoxycarbonyl, —CO₂R⁴ or tetrazolyl group.

In a further aspect in which R¹ represents —X-aryl or —X-heterocyclyl, the X-aryl group is other than benzyl and the X-heterocyclyl group is other than N-phthalimidoalkyl.

In one aspect, R¹ represents:
—X-heteroaryl (e.g. -pyridinyl) optionally substituted by a —CONR⁶R⁷ (e.g. —CONHMe) group or a halogen atom (e.g. Br); or
—X-heteroaryl-Y-heterocyclyl (e.g. pyridinyl-pyrrolidinyl) optionally substituted by an oxo group.

In a more particular aspect, R¹ represents —X-heteroaryl (e.g. -pyridinyl) optionally substituted by a —CONR⁶R⁷ (e.g. —CONHMe) group.

The term ‘C_{x-y} alkyl’ as used herein as a group or a part of the group refers to a linear or branched saturated hydrocarbon group containing from x to y carbon atoms. Examples of C₁₋₆ alkyl groups include methyl, ethyl, n-propyl, isopropyl, n-butyl, isobutyl, sec-butyl, tert butyl, n-pentyl, isopentyl, neopentyl or hexyl and the like.

The term ‘C_{x-y} alkenyl’ as used herein refers to a linear or branched hydrocarbon group containing one or more carbon-carbon double bonds and having from x to y carbon atoms. Examples of C₂₋₆ alkenyl groups include ethenyl, propenyl, butenyl, pentenyl or hexenyl and the like.

The term ‘C_{x-y} alkoxy’ as used herein refers to an —O—C_{x-y} alkyl group wherein C_{x-y} alkyl is as defined herein. Examples of C₁₋₆ alkoxy groups include methoxy, ethoxy, propoxy, butoxy, pentoxy or hexoxy and the like.

The term ‘C_{x-y} cycloalkyl’ as used herein refers to a saturated monocyclic hydrocarbon ring of x to y carbon atoms. Examples of C₃₋₈ cycloalkyl groups include cyclopropyl, cyclobutyl, cyclopentyl, cyclohexyl, cycloheptyl or cyclooctyl and the like.

The term ‘halogen’ as used herein refers to a fluorine, chlorine, bromine or iodine atom.

The term ‘haloC_{x-y} alkyl’ as used herein refers to a C_{x-y} alkyl group as defined herein wherein at least one hydrogen atom is replaced with halogen. Examples of haloC₁₋₆ alkyl groups include fluoroethyl, trifluoromethyl or trifluoroethyl and the like.

The term ‘haloC_{x-y} alkoxy’ as used herein refers to a C_{x-y} alkoxy group as herein defined wherein at least one hydrogen atom is replaced with halogen. Examples of haloC₁₋₆ alkoxy groups include difluoromethoxy or trifluoromethoxy and the like.

The term ‘aryl’ as used herein refers to a C₆₋₁₂ monocyclic or bicyclic hydrocarbon ring wherein at least one ring is aromatic. Examples of such groups include phenyl, naphthyl or tetrahydronaphthalenyl and the like.

The term ‘aryloxy’ as used herein refers to an —O-aryl group wherein aryl is as defined herein. Examples of such groups include phenoxy and the like.

The term ‘heteroaryl’ as used herein refers to a 5-6 membered monocyclic aromatic or a fused 8-10 membered bicyclic aromatic ring, which monocyclic or bicyclic ring contains 1 to 4 heteroatoms selected from oxygen, nitrogen and sulphur. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl and the like. Examples of such fused aromatic rings include quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridinyl, furopyridinyl, benzofuranyl, isobenzofuranyl, benzothienyl, benzoimidazolyl, benzoxazolyl, benzoisoxazolyl, benzothiazolyl, benzoisothiazolyl, benzoxadiazolyl, benzothiadiazolyl and the like. In one aspect, the term ‘heteroaryl’ refers to a 6 membered monocyclic aromatic ring.

The term ‘heterocyclyl’ refers to a 4-7 membered monocyclic ring or a fused 8-12 membered bicyclic ring which may be saturated or partially unsaturated and which monocyclic or bicyclic ring contains 1 to 4 heteroatoms selected from oxygen, nitrogen or sulphur. Examples of such monocyclic rings include pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl, azepanyl and the like. Examples of such bicyclic rings include indolinyl, isoindolinyl, benzopyranyl, quinuclidinyl, 2,3,4,5-tetrahydro-1H-3-benzazepine, tetrahydroisoquinolinyl and the like.

In one embodiment, R¹ represents:
—X-aryl (e.g. phenyl);
—X-aryl-Y-heterocyclyl (e.g. phenyl-pyrrolidinyl);
—X-heterocyclyl-Y-heterocyclyl (e.g. piperidinyl-CO-morpholinyl);
—X-heteroaryl e.g. pyridinyl or pyrazinyl); or
—X-heteroaryl-Y-heterocyclyl (e.g. pyridinyl-pyrrolidinyl, pyridinyl-CO-pyrrolidinyl, pyridinyl-imidazolidinyl or pyridinyl-oxazolidinyl).

In one aspect, the aryl, heteroaryl or heterocyclic groups of R¹ may optionally be substituted by one or more (e.g. 1, 2 or 3) substituents which may be the same or different, and which are selected from the group consisting of halogen, cyano, oxo, nitro, —R⁴, —OR⁴, —COR⁴, —CO₂R⁴, —NR⁵R⁶, —CONR⁵R⁶, —NR⁵COR⁶ and —SO₂R⁷, wherein R⁴, R⁵ and R⁶ independently represent H or —C₁₋₆ alkyl, and wherein R⁷ represents —C₁₋₆ alkyl.

In a more particular aspect, the aryl, heteroaryl or heterocyclic groups of R¹ may optionally be substituted by one or more (e.g. 1, 2 or 3) substituents which may be the same or different, and which are selected from the group consisting of halogen, cyano, oxo, —R⁴, —OR⁴, —CO₂R⁴, —CONR⁵R⁶ and —NR⁵COR⁶, wherein R⁴, R⁵ and R⁶ independently represent H or —C₁₋₆ alkyl.

In a more particular embodiment, R¹ represents:
—X-aryl (e.g. phenyl) optionally substituted by a halogen atom (e.g. Br);
—X-aryl-Y-heterocyclyl (e.g. phenyl-pyrrolidinyl) optionally substituted by an oxo group; —X-heteroaryl (e.g. -pyridinyl or -pyrazinyl) optionally substituted by a —CONR⁶R⁷ (e.g. —CONH₂, —CONHMe, —CONHEt, —CON(Me)₂, —CONH(1-methylethyl)) group, a —CO₂R⁴ group (e.g. —CO₂H or —CO₂Me) and/or a halogen atom (e.g. Br or I); or
—X-heteroaryl-Y-heterocyclyl (e.g. pyridinyl-pyrrolidinyl, pyridinyl-CO-pyrrolidinyl, pyridinyl-imidazolidinyl or pyridinyl-oxazolidinyl) optionally substituted by an oxo group and/or an —R⁴ (e.g. methyl) group.

More particularly, R¹ represents:
—X-aryl (e.g. phenyl) optionally substituted by a halogen atom (e.g. Br);
—X-aryl-Y-heterocyclyl (e.g. phenyl-N-pyrrolidinyl) optionally substituted on the pyrrolidinyl group by an oxo group (e.g. phenyl-N-pyrrolidin-2-one);
—X-heteroaryl (e.g. 2-pyridinyl or 2-pyrazinyl) optionally substituted by a —CONR⁶R⁷ (e.g. —CONH₂, —CONHMe, —CONHEt, —CON(Me)₂, —CONH(1-methylethyl)) group, a —CO₂R⁴ group (e.g. —CO₂H or —CO₂Me) or a halogen atom (e.g. Br or I); or
—X-heteroaryl-Y-heterocyclyl (e.g. 2-pyridinyl-N-pyrrolidinyl, 2-pyridinyl-CO—N-pyrrolidinyl, 2-pyridinyl-N-imidazolidinyl or 2-pyridinyl-N-oxazolidinyl) optionally substituted on the heterocyclic group by an oxo group (e.g. 2-pyridinyl-N-pyrrolidin-2-one, 2-pyridinyl-N-imidazolidin-2-one or 2-pyridinyl-N-oxazolidin-2-one) and/or an —R⁴ (e.g. methyl) group.

Even more particularly, R¹ represents:
—X-heteroaryl (e.g. 2-pyridinyl or 2-pyrazinyl) optionally substituted by a —CONR⁶R⁷ (e.g. —CONH₂, —CONHMe, —CONHEt, —CON(Me)₂, —CONH(1-methylethyl)) group, a —CO₂R⁴ group (e.g. —CO₂H or —CO₂Me) or a halogen atom (e.g. Br or I); or
—X-heteroaryl-Y-heterocyclyl (e.g. 2-pyridinyl-N-pyrrolidinyl, 2-pyridinyl-CO—N-pyrrolidinyl, 2-pyridinyl-N-imidazolidinyl or 2-pyridinyl-N-oxazolidinyl) optionally substituted on the heterocyclic group by an oxo group (e.g. 2-pyridinyl-N-pyrrolidin-2-one, 2-pyridinyl-N-imidazolidin-2-one or 2-pyridinyl-N-oxazolidin-2-one) and/or an —R⁴ (e.g. methyl) group.

More particularly, R¹ represents:
N-methylpyridin-2-yl 5-carboxamide;
5-(1-pyrrolidin-2-one)pyridin-2-yl;
5-(3-methyl-1-imidazolidin-2-one)pyridin-2-yl;
5-(1-oxazolidin-2-one)pyridin-2-yl; or
5-(1-pyrrolidinylcarbonyl)pyridin-2-yl.

Most particularly, R¹ represents 5-(1-pyrrolidin-2-one)pyridin-2-yl.

In one embodiment in which R¹ represents —X-aryl or —X-heteroaryl, wherein the aryl and heteroaryl groups are six membered rings that are substituted by one substitutent, the substituent is in the para position relative to the attachment to X.

In another embodiment in which R¹ represents —X-aryl-Y-heterocyclyl or —X-heteroaryl-Y-heterocyclyl, wherein the aryl and heteroaryl groups are six membered rings, the bond to Y is para to the bond to X.

In a further embodiment in which R¹ represents —X-aryl-Y-heterocyclyl or —X-heteroaryl-Y-heterocyclyl, wherein the heterocyclic group contains nitrogen, the atom in the heterocyclic group that links to Y is nitrogen.

In another embodiment, X represents a bond.

In a further embodiment, Y represents a bond or CO. More particularly, Y represents a bond.

In yet another embodiment, m represents 0 or 1. In a more particular embodiment, m represents 0.

In certain embodiments in which R² is present, R² represents a halogen atom or cyano group.

In one embodiment, n represents an integer from 2 to 4. More particularly, n represents 2.

In a further embodiment, p represents an integer from 0 to 2. More particularly, p represents 0 or 1, and most particularly, p represents 0.

In one embodiment, R³ represents —C₁₋₃ alkyl, particularly methyl.

Compounds of formula (I) may exist as stereoisomers in which the 2 position of the indenyl ring is a chiral centre. In one embodiment, the compounds of the invention include a single enantiomer, for example, the (−) enantiomer.

In one aspect, the invention provides a compound of formula (I) or a pharmaceutically acceptable salt thereof, wherein:

R¹ represents —X-heteroaryl, —X-heteroaryl-Y-heterocyclyl, X-aryl, —X-aryl-Y-heterocyclyl or —X-heterocyclyl-Y-heterocyclyl;
X represents a bond;
Y represents a bond or CO;
R² represents halogen or cyano;
m represents 0 or 1;
n represents 2;
p represents 0;
wherein said aryl, heteroaryl and heterocyclyl groups of R¹ may be optionally substituted by one or more substituents (e.g. 1, 2 or 3) which may be the same or different, and which are selected from the group consisting of halogen, cyano, oxo, —R⁴, —OR⁴, —CONR⁵R⁶ and —NR⁵COR⁶, wherein R⁴, R⁵ and R⁶ independently represent —C₁₋₆ alkyl;
or solvates thereof.

In a more particular aspect, the invention provides a compound of formula (I) or a pharmaceutically acceptable salt thereof, wherein:

R¹ represents —X-heteroaryl or —X-heteroaryl-Y-heterocyclyl;
X represents a bond;
Y represents a bond;
R² represents halogen or cyano;
m represents 0 or 1;
n represents 2;
p represents 0;
wherein said heteroaryl and heterocyclyl groups of R¹ may be optionally substituted by one or more substituents (e.g. 1, 2 or 3) which may be the same or different, and which are selected from the group consisting of halogen, cyano, oxo, —R⁴, —OR⁴, —CONR⁵R⁶ and —NR⁵COR⁶, wherein R⁴, R⁵ and R⁶ independently represent —C₁₋₆ alkyl;
or solvates thereof.

Compounds according to the invention include the compounds of examples E1-E27 as shown below, or pharmaceutically acceptable salts or solvates thereof.

More particularly, compounds of the invention include:
1-(6-{[2-(1-Pyrrolidinyl)-2,3-dihydro-1H-inden-5-yl]oxy}3-pyridinyl)-2-pyrrolidinone, particularly the (−) enantiomer;
5-(1-Pyrrolidinylcarbonyl)-2-{[2-(1-pyrrolidinyl)-2,3-dihydro-1H-inden-5-yl]oxy}pyridine;
1-[6-({2-[(2S)-2-Methyl-1-pyrrolidinyl]-2,3-dihydro-1H-inden-5-yl}oxy)-3-pyridinyl]-2-pyrrolidinone;
N-methyl-6-{[2-(1-pyrrolidinyl)-2,3-dihydro-1H-inden-5-yl]oxy}-3-pyridinecarboxamide, particularly the (−) enantiomer;
1-Methyl-3-(6-{[2-(1-pyrrolidinyl)-2,3-dihydro-1H-inden-5-yl]oxy}-3-pyridinyl)-2-imidazolidinone; and
3-(6-{[2-(1-Pyrrolidinyl)-2,3-dihydro-1H-inden-5-yl]oxy}-3-pyridinyl)-1,3-oxazolidin-2-one;
   or pharmaceutically acceptable salts or solvates thereof.

Most particularly, compounds of the invention include 1-(6-{[2-(1-pyrrolidinyl)-2,3-dihydro-1H-inden-5-yl]oxy}-3-pyridinyl)-2-pyrrolidinone, particularly the (−) enantiomer, or pharmaceutically acceptable salts or solvates thereof.

Because of their potential use in medicine, the salts of the compounds of formula (I) are preferably pharmaceutically acceptable.

A pharmaceutically acceptable acid addition salt can be formed by reaction of a compound of formula (I) with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

The invention includes within its scope all possible stoichiometric and non-stoichiometric forms of the salts of the compounds of formula (I) including hydrates and solvates. Compounds of formula (I) are capable of existing in stereoisomeric forms. It will be understood that the invention encompasses all geometric and optical isomers of these compounds and the mixtures thereof including racemates. Tautomers also form an aspect of the invention.

The present invention also provides a process for the preparation of a compound of formula (I) or a pharmaceutically acceptable salt thereof, which process comprises:

(a) reacting a compound of formula (II)

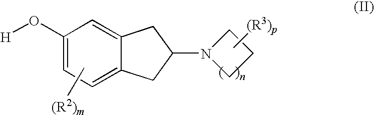

wherein R², R³, m, n and p are as defined above, with a compound of formula R^{1′}-L¹, wherein R^{1′} is as defined above for R¹ or a group convertible thereto and L¹ represents a suitable leaving group such as a halogen atom (e.g. chlorine, bromine or iodine) or a hydroxyl group;
(b) reacting a compound of formula (II)

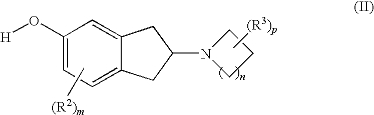

wherein R², R³, m, n and p are as defined above, with a compound of formula R^{1′}—X¹, wherein R^{1′} is as defined above for R¹ or a group convertible thereto and X¹ represents a boronic acid group;
(c) reacting a compound of formula (III)

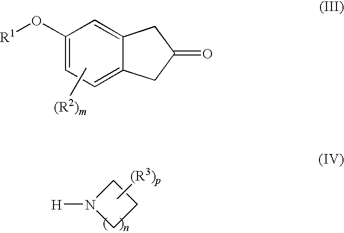

wherein R¹, R² and m are as defined above, with a compound of formula (IV) wherein R³, n and p are as defined above; or
(d) deprotecting a compound of formula (I) which is protected;
(e) interconversion from one compound of formula (I) to another; and
(f) separation of a racemic mixture of a compound of formula (I) to produce a stereoisomer of a compound of formula (I).

When the leaving group L¹ is attached to an sp³ hybridised carbon, for example, R^{1′}-L¹ is an alkyl halide, process (a) typically comprises the use of a suitable base, such as potassium carbonate in an appropriate solvent such as 2-butanone optionally in the presence of a catalyst such as potassium iodide at an appropriate temperature such as reflux.

When L¹ is a hydroxyl group attached to an sp³ hybridised carbon, for example, R^{1′}-L¹ is an alcohol, process (a) typically comprises the use of a phosphine such as triphenylphosphine in a suitable solvent such as tetrahydrofuran, followed by addition of an azodicarboxylate such as diethylazodicarboxylate at a suitable temperature such as room temperature.

When the leaving group L¹ is attached to an sp² hybridised carbon, for example, R^{1′}-L¹ is an aryl halide or heteroaryl halide, process (a) typically comprises the use of a copper(I) salt, such as copper (I) iodide, in the presence of a base such as sodium hydride, in an appropriate solvent such as pyridine, at an appropriate temperature such as reflux.

When the leaving group L¹ is attached to an activated sp² hybridised carbon for example, R^{1′}-L¹ is a heteroaryl halide such as a 2-chloropyridine or 2-chloropyrazine, process (a) typically comprises the use of a suitable base, such as sodium hydride or potassium carbonate in an appropriate solvent such as dimethylformamide or dimethyl sulfoxide, at an appropriate temperature, such as between 80-90° C. or 150° C. Alternatively, potassium tert-butoxide in tert-butanol at an appropriate temperature may also be employed.

When the leaving group L¹ is attached to an activated sp² hybridised carbon, for example R^{1′}-L¹ is an aryl halide such as 3,4-difluoro-benzonitrile, process (a) typically comprises the use of a suitable base, potassium carbonate, in a suitable solvent, such as dimethyl sulfoxide, at a suitable temperature.

Process (b) typically comprises the use of a suitable base such as triethylamine in an appropriate solvent such as dichloromethane at a suitable temperature such as room temperature.

Process (c) typically comprises the use of reductive conditions (such as treatment with a borohydride e.g. sodium triacetoxyborohydride), optionally In the presence of an acid, such as acetic acid, in an appropriate solvent such as dichloromethane at a suitable temperature such as between room temperature and 40° C.

In process (d), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid in dioxan or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Process (e) may be performed using conventional interconversion procedures such as epimerisation, oxidation, reduction, alkylation, nucleophilic or electrophilic aromatic substitution, ester hydrolysis, amide bond formation or transition metal mediated coupling reactions. Examples of transition metal mediated coupling reactions useful as interconversion procedures include the following: Palladium catalysed coupling reactions between organic electrophiles, such as aryl halides, and organometallic reagents, for example boronic acids (Suzuki cross-coupling reactions); Palladium catalysed amination and amidation reactions between organic electrophiles, such as aryl halides, and nucleophiles, such as amines and amides; Copper catalysed amidation reactions between organic electrophiles (such as aryl halides) and nucleophiles such as amides; and Copper mediated coupling reactions between phenols and boronic acids.

Process (f) may be performed by conventional separation techniques such as chiral chromatography, for example using a Chiralcel OD column eluting with a 1-1 mixture of heptane-ethanol.

Compounds of formula (II) and (III) may be prepared in accordance with the following scheme:

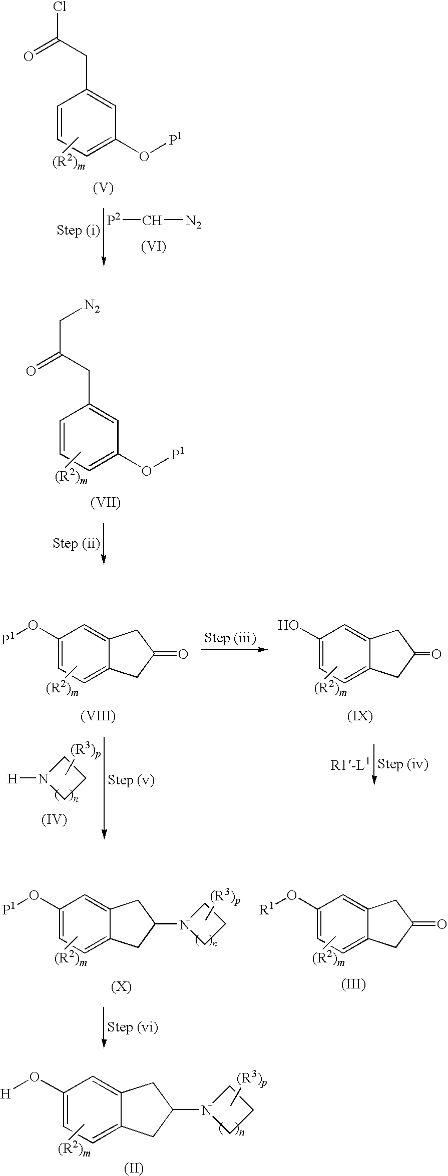

wherein R¹, R¹, R², R³, m, n, p and L¹ are as defined above, P¹ represents a suitable protecting group such as methyl and P² represents either hydrogen or trimethylsilyl.

Step (i) comprises reaction with a compound of formula (VI) at a suitable temperature such as room temperature, in a suitable solvent such as a 1:1 mixture of tetrahydrofuran:acetonitrile.

Step (ii) typically comprises treatment with rhodium (II) acetate dimer dihydrate in a suitable solvent such as dichloromethane, at a suitable temperature such as between room temperature and 40° C.

Step (iii) typically comprises a deprotection reaction, for example, when P¹ represents methyl a compound of formula (VIII) can be deprotected using boron tribromide in dichloromethane at a suitable temperature, such as room temperature. Alternatively, when P¹ represents methyl, a compound of formula (VIII) can be deprotected by refluxing in hydrobromic acid.

Step (iv) may be performed in an analogous manner to that described for process (a).

Step (v) may be performed in an analogous manner to that described for process (c).

Step (vi) typically comprises a deprotection reaction to provide a compound of formula (II) and can be performed as described in step (iii).

Compounds of formula (IV), (V), R^{1′}-L¹ and R^{1′}—X¹ are either commercially available or can be prepared in accordance with known literature procedures.

Compounds of formula (I) and their pharmaceutically acceptable salts have affinity for and are antagonists and/or inverse agonists of the histamine H3 receptor and are believed to be of potential use in the treatment of neurological diseases including Alzheimer's disease, dementia (including Lewy body dementia and vascular dementia), age-related memory dysfunction, mild cognitive impairment, cognitive deficit, epilepsy, pain of neuropathic origin including neuralgias, neuritis and back pain, and inflammatory pain including osteoarthritis, rheumatoid arthritis, acute inflammatory pain and back pain, migraine, Parkinson's disease, multiple sclerosis, stroke and sleep disorders (including narcolepsy and sleep deficits associated with Parkinson's disease); psychiatric disorders including schizophrenia (particularly cognitive deficit of schizophrenia), attention deficit hyperactivity disorder, depression, anxiety and addiction; and other diseases including obesity and gastro-intestinal disorders.

It will also be appreciated that compounds of formula (I) are expected to be selective for the histamine H3 receptor over other histamine receptor subtypes, such as the histamine H1 receptor. Generally, compounds of the invention may be at least 10 fold selective for H3 over H1, such as at least 100 fold selective.

Thus the invention also provides a compound of formula (I) or a pharmaceutically acceptable salt thereof, for use as a therapeutic substance in the treatment or prophylaxis of the above disorders, in particular cognitive impairments in diseases such as Alzheimer's disease and related neurodegenerative disorders.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment of the above disorders.

When used in therapy, the compounds of formula (I) are usually formulated in a standard pharmaceutical composition. Such compositions can be prepared using standard procedures.

Thus, the present invention further provides a pharmaceutical composition for use in the treatment of the above disorders which comprises the compound of formula (I) or a pharmaceutically acceptable salt thereof and a pharmaceutically acceptable carrier.

The present invention further provides a pharmaceutical composition which comprises the compound of formula (I) or a pharmaceutically acceptable salt thereof and a pharmaceutically acceptable carrier.

Compounds of formula (I) may be used in combination with other therapeutic agents, for example medicaments claimed to be useful as either disease modifying or symptomatic treatments of Alzheimer's disease. Suitable examples of such other therapeutic agents may be agents known to modify cholinergic transmission such as 5-HT₆ antagonists, M1 muscarinic agonists, M2 muscarinic antagonists or acetylcholinesterase inhibitors. When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound. The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.1 to 200 mg and even more suitably 1.0 to 200 mg, and such unit doses may be administered more than once a day, for example two or three a day. Such therapy may extend for a number of weeks or months.

The following Descriptions and Examples illustrate the preparation of compounds of the invention.

Hydrochloride salts of the compounds of the invention may be prepared by standard methods. For example, a free base may be converted into the corresponding hydrochloride salt by treatment in methanol with a solution of hydrogen chloride in diethyl ether followed by evaporation of solvents.

Where indicated, Mass Directed Auto-Purification or MDAP was carried out using a Supelco LCABZ++ column (20 mm×100 mm). The stationary phase particle size is 5 μm. The solvent systems used comprised solvent A (water+0.1% formic acid) and solvent B (acetonitrile:water 95:5+0.05% formic acid). Compounds were eluted with gradients of solvent B in solvent A.

### Description 1

### 1-Diazo-3-[3-methyloxy)phenyl]-2-propanone (D1)

[3-(Methyloxy)phenyl]acetyl chloride (3.99 g, 3.37 ml, 21.6 mmol) was dissolved in tetrahydrofuran (20 ml) and acetonitrile (20 ml) and cooled to 0° C. under argon with stirring. 2M (Trimethylsilyl)diazomethane in hexane solution was added dropwise. The mixture was allowed to warm to room temperature and stirred for 18 hours. The solvent was evaporated to leave the title compound (D1) as an orange oil. (4.2 g, assumed 100%); MS m/e 191 [M+H]⁺.

### Description 2

### 5-(Methyloxy)-1,3-dihydro-2H-inden-2-one (D2)

1-Diazo-3-[3-(methyloxy)phenyl]-2-propanone (4.2 g, assumed 21.6 mmol; may be prepared as described in Description 1) was dissolved in dichloromethane (60 ml) and rhodium(II) acetate dimer dihydrate (516 mg, 1.08 mmol) added. After nitrogen evolution had ceased the solution was heated at 40° C. for 2 hours. The mixture was evaporated and the residue purified by flash chromatography on silica gel eluting with a mixture of n-pentane and ethyl acetate (80:20) to give the title compound (D2). (1.30 g, 37%); MS m/e 163 [M+H]⁺.

Impure fractions from the chromatography were further purified by flash chromatography on silica gel eluting with a mixture of n-pentane and ethyl acetate (85:15) to give a second crop of the title compound (D2). (680 mg, 19%); MS m/e 163 [M+H]⁺.

### Description 3

### 1-[5-(Methyloxy)-2,3-dihydro-1H-inden-2-yl]pyrrolidine (D3)

### Method A

A mixture of 5-(methyloxy)-1,3-dihydro-2H-inden-2-one (may be prepared as described in Description 2) (60 mg, 0.37 mmol), pyrrolidine (31 mg, 0.41 mmol) and acetic acid (1 drop, catalytic amount) in dichloromethane (5 ml) was stirred at room temperature for 20 minutes. Sodium triacetoxyborohydride was then added and the mixture stirred for 18 hours. The reaction was then diluted with methanol, applied to an scx ion exchange column and eluted with methanol and then a solution of ammonia in methanol (2M). The basic fractions were reduced and the residue chromatographed on silica gel eluting with a mixture of ammonia in methanol and dichloromethane (2:98) to afford the title compound (D3) (40 mg, 50%); ¹H NMR (CDCl₃) 7.08 (1H, d), 6.75 (1H, s), 6.69 (1H, d), 3.78 (3H, s), 2.95 (5H, m), 2.6 (4H, m), 1.78 (4H, m).

### Method B

5-(Methyloxy)-1,3-dihydro-2H-inden-2-one (may be prepared as described in Description 2) (1.5 g, 9.25 mmol) was dissolved in dichloromethane (20 ml) and treated with pyrrolidine (1.54 ml, 18.5 mmol) and acetic acid (1 drop, catalytic amount). The mixture was cooled in an ice bath and sodium triacetoxyborohydride (3.9 g, 18.5 mmol) was added portionwise. The resulting mixture was stirred at room temperature for 3.5 hours. The reaction was then diluted with methanol, applied to an scx ion exchange column and eluted with methanol and then a solution of ammonia in methanol (2M). The basic fractions were combined and evaporated under reduced pressure and the residue chromatographed on silica gel eluting with a mixture of 2M ammonia in methanol and dichloromethane (2.5:97.5 to 10:90) to afford the title compound (D3); MS (ES+) m/e 218 [M+H]⁺.

### Method C

A mixture of 5-(methyloxy)-1,3-dihydro-2H-inden-2-one (may be prepared as described in Description 2; 1.30 g, 8 mmol), and acetic acid (5 ml) in dichloromethane (50 ml) was stirred at 0° C. and pyrrolidine (1.14 g, 1.32 ml, 16 mmol) added. The mixture was stirred and allowed to reach room temperature over 15 minutes. Sodium triacetoxyborohydride (3.38 g, 16 mmol) was then added portionwise and the mixture stirred at room temperature for 2 hours. The reaction was washed with water and the aqueous layer extracted with dichloromethane (×2). The combined organic layers were dried over magnesium sulphate and evaporated. The brown oil was diluted with methanol then applied to an SCX ion exchange column and eluted with methanol and then a solution of ammonia in methanol (2M). The basic fractions were reduced to afford the title compound (D3) (1.24 g, 71%); MS m/e 218 [M+H]⁺.

### Description 4

### 2-(1-Pyrrolidinyl)-2,3-dihydro-1H-inden-5-ol (D4)

### Method A

1-[5-(Methyloxy)-2,3-dihydro-1H-inden-2-yl]pyrrolidine (may be prepared as described in Description 3) (40 mg, 0.18 mmol) in dichloromethane (2 ml) and treated dropwise with boron tribromide (0.37 ml, 0.37 mmol). The solution was stirred at room temperature for 18 hours and then quenched with water. The mixture was stirred for 20 minutes and then diluted with methanol, applied to an scx ion exchange column and eluted with methanol and then a solution of ammonia in methanol (2M). The basic fractions were reduced and the residue chromatographed on silica gel eluting with a mixture of ammonia in methanol and dichloromethane (3:97) to afford the title compound (D4) (15 mg, 41%); MS (ES+) m/e 204 [M+H]⁺.

### Method B

Boron tribromide (1M solution in dichloromethane) (11.4 ml, 11.4 mmol) was added dropwise to a solution of 1-[5-(methyloxy)-2,3-dihydro-1H-inden-2-yl]pyrrolidine (may be prepared as described in Description 3) (1.24 g, 5.71 mmol) in dichloromethane (15 ml). The resulting mixture was stirred at room temperature under argon for 5 hours. The mixture was diluted with methanol, applied to an scx ion exchange column and eluted with methanol and then a solution of ammonia in methanol (2M). The basic fractions were evaporated under reduced pressure to afford the title compound (D4); MS (ES+) m/e 204 [M+H]⁺.

### Method C

1-[5-(Methyloxy)-2,3-dihydro-1H-inden-2-yl]pyrrolidine (1.238 g, 5.7 mmol; may be prepared as described in Description 3) was dissolved in 48% aqueous hydrobromic acid (20 ml) and the solution heated at reflux with stirring for 2 hours. After cooling the solution was evaporated and the residue re-evaporated from toluene (×3). The brown oil was purified on a 10 g SCX ion exchange cartridge, eluting with methanol then 2M ammonia in methanol. The basic fractions were evaporated and the residue further purified by flash chromatography on silica gel eluting with a mixture of 2M ammonia in methanol and dichloromethane (4:96-10:90) to afford the title compound (D4) (550 mg, 48%); MS m/e 204 [M+H]⁺.

### Description 5

### 1-[5-(Methyloxy)-2,3-dihydro-1H-inden-2-yl]hexahydro-1H-azepine (D5)

A mixture of 5-(methyloxy)-1,3-dihydro-2H-inden-2-one (may be prepared as described in Description 2) (150 mg, 0.93 mmol), hexahydro-1H-azepine (0.209 ml, 1.85 mmol), acetic acid (1 drop, catalytic amount) and sodium triacetoxyborohydride (392 mg, 1.85 mmol) in dichloromethane (5 ml) was stirred at room temperature for 18 hours. The reaction was then diluted with methanol, applied to an scx ion exchange column and eluted with methanol and then a solution of ammonia in methanol (2M). The basic fractions were combined and evaporated under reduced pressure and the residue chromatographed on silica gel eluting with a mixture of 2M ammonia in methanol and dichloromethane (2:98) to afford the title compound (D5); MS (ES+) m/e 246 [M+H]⁺.

### Description 6

### (2R,5R)-2,5-Dimethyl-1-[5-methyloxy)-2,3-dihydro-1H-inden-2-yl]pyrrolidine (D6)

A mixture of 5-(methyloxy)-1,3-dihydro-2H-inden-2-one (may be prepared as described in Description 2) (150 mg, 0.93 mmol), (2R,5R)-2,5-dimethylpyrrolidine hydrochloride (251 mg, 1.85 mmol), triethylamine (0.256 ml, 1.85 mmol) and acetic acid (1 drop, catalytic amount) in dichloromethane (5 ml) was stirred at room temperature for 30 minutes. Sodium triacetoxyborohydride (392 mg, 1.85 mmol) was added and the mixture stirred at 40° C. under argon for 4.5 hours. The reaction was then diluted with methanol, applied to an scx ion exchange column and eluted with methanol and then a solution of ammonia in methanol (2M). The basic fractions were combined and evaporated under reduced pressure to afford the title compound (D6); MS (ES+) m/e 246 [M+H]⁺.

### Description 7

### 2-(Hexahydro-1H-azepin-1-yl)-2,3-dihydro-1H-inden-5-ol (D7)

Boron tribromide (1M solution in dichloromethane) (2.6 ml, 2.6 mmol) was added dropwise to a solution of 1-[5-(methyloxy)-2,3-dihydro-1H-inden-2-yl]hexahydro-1H-azepine (may be prepared as described in Description 5) (320 mg, 1.3 mmol) in dichloromethane (3 ml). The resulting mixture was stirred at room temperature under argon for 4 hours. The mixture was diluted with methanol, applied to an scx ion exchange column and eluted with methanol and then a solution of ammonia in methanol (2M). The basic fractions were evaporated under reduced pressure to afford the title compound (D7); MS (ES+) m/e 232 [M+H]⁺.

### Description 8

### 2-[(2R,5R)-2,5-Dimethyl-1-pyrrolidinyl]-2,3-dihydro-1H-inden-5-ol (D8)

Boron tribromide (1M solution in dichloromethane) (1.22 ml, 1.22 mmol) was added dropwise to a solution of (2R,5R)-2,5-dimethyl-1-[5-(methyloxy)-2,3-dihydro-1H-inden-2-yl]pyrrolidine (may be prepared as described in Description 6) (150 mg, 0.61 mmol) in dichloromethane (3 ml). The resulting mixture was stirred at room temperature under argon for 1.5 hours. The mixture was diluted with methanol, applied to an scx ion exchange column and eluted with methanol and then a solution of ammonia in methanol (2M). The basic fractions were evaporated under reduced pressure to afford the title compound (D8); MS (ES+) m/e 232 [M+H]⁺.

### Description 9

### 2-Chloro-5-(1-pyrrolidinylcarbonyl)pyridine (D9)

Pyrrolidine (0.533 ml, 6.4 mmol) was added to a solution of 6-chloro-3-pyridinecarbonyl chloride (350 mg, 1.99 mmol) in dichloromethane (6 ml). The resulting mixture was stirred at room temperature for 24 hours. The reaction mixture was diluted with water (15 ml) and extracted with dichloromethane (×3). The dichloromethane layers were combined, dried under magnesium sulfate and evaporated under reduced pressure to afford the title compound (D9); MS (ES+) m/e 211 [M+H]⁺.

### Descriptions 10-11 (D10-D11)

The following descriptions were prepared from 6-chloro-3-pyridinecarbonyl chloride and the corresponding amine using an analogous method to that described in Description 9:

| | | MS (ES+) m/e |
|---|---|---|
| Description | Amine | [M + H]⁺ |
| 6-Chloro-3- | Ammonia | 157 |
| pyridinecarboxamide | | |
| (D10) | | |
| 6-Chloro-N-(1- | Isopropylamine | 199 |
| methylethyl)-3- | | |
| pyridinecarboxamide | | |
| (D11) | | |

### Description 12

### 6-Chloro-N-ethyl-3-pyridinecarboxamide (D12)

Ethylamine (2M in tetrahydrofuran) (3.2 ml, 6.4 mmol) was added to a solution of 6-chloro-3-pyridinecarbonyl chloride (350 mg, 1.99 mmol) in dichloromethane (6 ml). The resulting mixture was stirred at room temperature for 24 hours. Ethylamine (2M in tetrahydrofuran) (3.2 ml, 6.4 mmol) was added and the mixture stirred at room temperature for 72 hours. The reaction mixture was diluted with water (15 ml) and extracted with dichloromethane (×3). The dichloromethane layers were combined, dried under magnesium sulfate and evaporated under reduced pressure to afford the title compound (D12); MS (ES+) m/e 185 [M+H]⁺.

### Description 13

### 6-Chloro-N,N-dimethyl-3-pyridinecarboxamide (D13)

Dimethylamine (2M in tetrahydrofuran) (3.2 ml, 6.4 mmol) was added to a solution of 6-chloro-3-pyridinecarbonyl chloride (350 mg, 1.99 mmol) in dichloromethane (6 ml). The resulting mixture was stirred at room temperature for 24 hours. Dimethylamine (2M in tetrahydrofuran) (3.2 ml, 6.4 mmol) was added and the mixture stirred at room temperature for 72 hours. The reaction mixture was diluted with water (15 ml) and extracted with dichloromethane (×3). The dichloromethane layers were combined, dried under magnesium sulfate and evaporated under reduced pressure. The residue was chromatographed on silica gel eluting with a mixture of pentane and ethyl acetate (1:4) to afford the title compound (D13); MS (ES+) m/e 185 [M+H]⁺.

### Description 14

### 1-[5-(Methyloxy)-2,3-dihydro-1H-inden-2-yl]piperidine (D14)

A mixture of 5-(methyloxy)-1,3-dihydro-2H-inden-2-one (may be prepared as described in Description 2) (250 mg, 1.08 mmol), piperidine (213 μl, 2.16 mmol), sodium triacetoxyborohydride (458 mg, 2.16 mmol) and acetic acid (1 drop, catalytic amount) in dichloromethane (10 ml) was stirred at room temperature for 18 hours. The reaction was then diluted with methanol, applied to an SCX ion exchange column and eluted with methanol and then a solution of ammonia in methanol (2M). The basic fractions were combined and evaporated to give the title compound (D14) MS (ES+) m/e 231 [M+H]⁺.

### Description 15

### 2-(1-Piperidinyl)-2,3-dihydro-1H-inden-5-ol (D15)

A 1M solution of boron tribromide in dichloromethane (2.1 ml, 2.1 mmol) was added drop-wise to solution of 1-[5-(methyloxy)-2,3-dihydro-1H-inden-2-yl]piperidine (239 mg, 1.03 mmol; may be prepared as described in Description 14) in dichloromethane (2 ml) and the mixture stirred at room temperature for 3 hours. The mixture was purified on a 10 g SCX ion exchange column and eluted with methanol and then a solution of ammonia in methanol (2M). The basic fractions were combined and evaporated to give the title compound (D15) MS (ES+) m/e 218 [M+H]⁺.

### Descriptions 16-17 (D16-D17)

Descriptions 16 and 17 (D16 & D17) were prepared using an analogous method to that described in Description 14 from 5-(methyloxy)-1,3-dihydro-2H-inden-2-one (may be prepared as described in Description 2) and the appropriate amine, as shown in the table below:

| Product | Amine | MS Data |
|---|---|---|
| (2R)-2-Methyl-1-[5-(methyloxy)-2,3- | (2R)-2- | MS (ES+) m/e |
| dihydro-1H-inden-2-yl]pyrrolidine | methylpyrrolidine | 232 [M + H]⁺. |
| (D16) | | |
| (2S)-2-methyl-1-[5-(methyloxy)-2,3- | (2S)-2- | MS (ES+) m/e |
| dihydro-1H-inden-2-yl]pyrrolidine | methylpyrrolidine | 232 [M + H]⁺. |
| (D17) | | |

### Descriptions 18-19 (D18-D19)

Descriptions 18 and 19 (D18 & D19) were prepared using an analogous method to that described in Description 15 from appropriate starting material as shown in the table below:

| Product | Starting Material | MS Data |
|---|---|---|
| 2-[(2R)-2-Methyl- | (2R)-2-Methyl-1-[5- | MS (ES+) m/e |
| 1-pyrrolidinyl]- | (methyloxy)-2,3-dihydro-1H- | 218 [M + H]⁺. |
| 2,3-dihydro-1H-inden- | inden-2-yl]pyrrolidine (may | |
| 5-ol (D18) | be prepared as described in | |
| | Description 16) | |
| 2-[(2S)-2-Methyl- | (2S)-2-methyl-1-[5- | MS (ES+) m/e |
| 1-pyrrolidinyl]- | (methyloxy)-2,3-dihydro-1H- | 218 [M + H]⁺. |
| 2,3-dihydro-1H-inden- | inden-2-yl]pyrrolidine (may | |
| 5-ol (D19) | be prepared as described in | |
| | Description 17) | |

### EXAMPLE 1

### N-Methyl-6-{[2-(1-pyrrolidinyl)-2,3-dihydro-1H-inden-5-yl]oxy}-3-pyridinecarboxamide (E1)

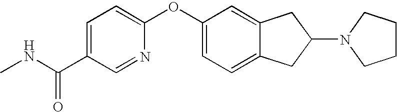

2-(1-Pyrrolidinyl-2,3-dihydro-1H-inden-5-ol (may be prepared as described in Description 4) (15 mg, 0.074 mmol) in dimethylformamide at room temperature was treated with sodium hydride (3.25 mg, 60% in mineral oil). After 20 minutes 6-chloro-N-methyl-3-pyridinecarboxamide (14 mg, 0.08 mmol; may be prepared as described in Description 10 of WO2004056369) was added and the mixture heated at 80° C. for 4 hours. The mixture was then cooled to room temperature, applied to an scx ion exchange column and eluted with methanol and then a solution of ammonia in methanol (2M). The basic fractions were reduced and the residue chromatographed on silica gel eluting with a mixture of ammonia in methanol and dichloromethane (4:96) to afford the title compound (E1) (11 mg, 44%); MS (ES+) m/e 338 [M+H]+.

### EXAMPLE 2

### 5-Bromo-2-{[2-(1-pyrrolidinyl)-2,3-dihydro-1H-inden-5-yl]oxy}pyridine (E2)

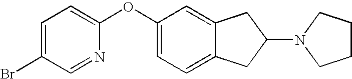

A solution of 2-(1-pyrrolidinyl)-2,3-dihydro-1H-inden-5-ol (may be prepared as described in Description 4) (162 mg, 0.8 mmol) in dry dimethylformamide (5 ml) was treated with sodium hydride (34 mg, 0.84 mmol) and the resulting mixture stirred at room temperature for 90 minutes. 5-Bromo-2-chloropyridine (308 mg, 1.6 mmol) was added and the mixture heated at 90° C. for 18 hours. The mixture was purified on a 5 g SCX ion exchange cartridge. The basic fractions were combined and evaporated to afford the title compound (E2). MS (AP+) m/e 359 & 361 [M+H]⁺.

### EXAMPLE 3

### 1-(6-{[2-(1-Pyrrolidinyl)-2,3-dihydro-1H-inden-5-yl]oxy}-3-pyridinyl)-2-pyrrolidinone (E3)

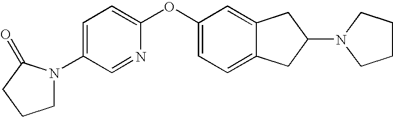

Copper I iodide (10 mg, 0.05 mmol) was added to a mixture of 5-bromo-2-{[2-(1-pyrrolidinyl)-2,3-dihydro-1H-inden-5-yl]oxy}pyridine (may be prepared as described in Example 2) (186 mg, 0.52 mmol), 2-pyrrolidinone (89 mg, 1.04 mmol), potassium carbonate (258 mg, 1.87 mmol) and N,N′-dimethyl-1,2-ethanediamine (5 mg, 0.05 mmol) in dioxan (5 ml) and the mixture was heated at reflux for 18 hours. The mixture was allowed to cool and was filtered through Celite. The filtrate was evaporated under reduced pressure and the residue purified by chromatography on silica gel eluting with a 1-19 mixture of 2M ammonia solution in methanol-dichloromethane to afford the title compound (E3) as a racemic mixture. MS (AP+) m/e 364 [M+H]⁺.

### EXAMPLE 4

### (−)-Enantiomer 1-(6-{[2-(1-Pyrrolidinyl)-2,3-dihydro-1H-inden-5-yl]oxy}-3-pyridinyl)-2-pyrrolidinone (E4)

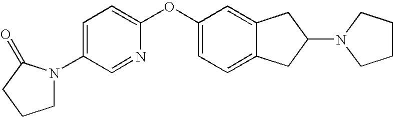

The racemic 1-(6-{[2-(1-pyrrolidinyl)-2,3-dihydro-1H-inden-5-yl]oxy}-3-pyridinyl)-2-pyrrolidinone (125 mg, 0.34 mmol) (may be prepared as described in Example 3) was separated on a 20 mm×250 mm 10 micron chiralcel OD column eluting with 1-1 heptane-ethanol at a flow rate of 17 ml/min. The fraction containing above enantiomer was evaporated under reduced pressure to obtain the title compound (E4) which had an [α]_{D}=−9.3° at 28.9° C. (MeOH). MS (AP+) m/e 364 [M+H]⁺. NMR (CDCl3) δ 8.27 (1H, m), 8.19 (1H, m), 7.18 (1H, m), 6.94-6.87 (3H, m), 3.84 (2H, t), 3.25-2.88 (5H, m), 2.80-2.55 (6H, m), 2.22 (2H, quintet), 1.86 (4H, m).

### EXAMPLE 5

### (+)-Enantiomer of 1-(6-{[2-(1-pyrrolidinyl)-2,3-dihydro-1H-inden-5-yl]oxy}-3-pyridinyl)-2-pyrrolidinone (E5)

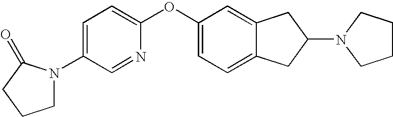

The racemic 1-(6-{[2-(1-pyrrolidinyl)-2,3-dihydro-1H-inden-5-yl]oxy}-3-pyridinyl)-2-pyrrolidinone (125 mg, 0.34 mmol) (may be prepared as described in Example 3) was separated on a 20 mm×250 mm 10 micron chiralcel OD column eluting with 1-1 heptane-ethanol at a flow rate of 17 ml/min. The fraction containing above enantiomer was evaporated under reduced pressure to obtain the title compound (E5) which had an [α]_{D}=+8.6° at 28.8° C. (MeOH). MS (AP+) m/e 364 [M+H]⁺. NMR (CDCl3) δ 8.28 (1H, m), 8.18 (1H, m), 7.18 (1H, m), 6.94-6.87 (3H, m), 3.85 (2H, t), 3.25-2.88 (5H, m), 2.80-2.55 (6H, m), 2.23 (2H, quintet), 1.87 (4H, m).

### EXAMPLE 6

### Methyl 5-{[2-(1-pyrrolidinyl)-2,3-dihydro-1H-inden-5-yl]oxy}-2-pyrazinecarboxylate (E6)

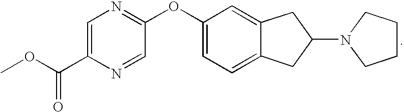

Sodium hydride (12 mg, 0.30 mmol, 60% in mineral oil) was added to a solution of 2-(1-pyrrolidinyl)-2,3-dihydro-1H-inden-5-ol (may be prepared as described in Description 4) (50 mg, 0.25 mmol) in dimethylformamide (3 ml) and the resulting mixture was stirred at room temperature for 20 minutes. Methyl 5-chloro-2-pyrazinecarboxylate (66 mg, 0.38 mmol) was added and the mixture heated at 90° C. under argon for 16 hours. The mixture was then cooled to room temperature, applied to an scx ion exchange column and eluted with methanol and then a solution of ammonia in methanol (2M). The basic fractions were evaporated under reduced pressure to afford the title compound (E6); MS (ES+) m/e 340 [M+H]+.

### EXAMPLE 7

### 5-{[2-(1-Pyrrolidinyl)-2,3-dihydro-1H-inden-5-yl]oxy}-2-pyrazinecarboxylic acid (E7)

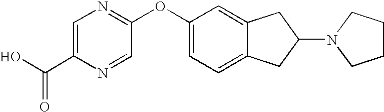

Methyl 5-{[2-(1-pyrrolidinyl)-2,3-dihydro-1H-inden-5-yl]oxy}-2-pyrazinecarboxylate (may be prepared as described in Example 6) (62 mg, 0.18 mmol) was dissolved in ethanol (3 ml), treated with 2M aqueous sodium hydroxide solution (0.28 ml, 0.55 mmol) and the resulting mixture was stirred at room temperature for 30 minutes. The mixture was diluted with methanol and applied to an scx ion exchange column and eluted with methanol and then a solution of ammonia in methanol (2M). The basic fractions were evaporated under reduced pressure to afford the title compound (E7); MS (ES+) m/e 326 [M+H]+.

### EXAMPLE 8

### 6-{[2-(Hexahydro-1H-azepin-1-yl)-2,3-dihydro-1H-inden-5-yl]oxy}-N-methyl-3-pyridinecarboxamide (E8)

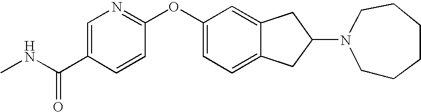

Sodium hydride (44 mg, 1.1 mmol, 60% in mineral oil) was added to a solution of 2-(hexahydro-1H-azepin-1-yl)-2,3-dihydro-1H-inden-5-ol (may be prepared as described in Description 7) (209 mg, 0.9 mmol) in dimethylformamide (4 ml) and the resulting mixture was stirred at room temperature for 20 minutes. 6-Chloro-N-methyl-3-pyridinecarboxamide (187 mg, 1.1 mmol; may be prepared as described in Description 10 of WO2004056369) was added and the mixture heated at 90° C. under argon for 18 hours. The mixture was then cooled to room temperature, applied to an scx ion exchange column and eluted with methanol and then a solution of ammonia in methanol (2M). The basic fractions were evaporated under reduced pressure and the residue chromatographed on silica gel eluting with a mixture of 2M ammonia in methanol and dichloromethane (5:95). The resulting compound was purified on the mass directed autoprep to afford the title compound (E8); MS (ES+) m/e 366 [M+H]+.

### EXAMPLE 9

### 6-({2-[(2R,5R)-2,5-dimethyl-1-pyrrolidinyl]-2,3-dihydro-1H-inden-5-yl}oxy)-N-methyl-3-pyridinecarboxamide (E9)

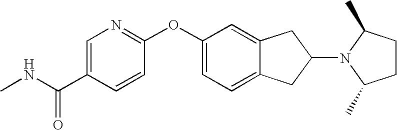

Sodium hydride (22 mg, 0.55 mmol, 60% in mineral oil) was added to a solution of 2-[(2R,5R)-2,5-dimethyl-1-pyrrolidinyl]-2,3-dihydro-1H-inden-5-ol (may be prepared as described in Description 8) (105 mg, 0.45 mmol) in dimethylformamide (4 ml) and the resulting mixture was stirred at room temperature for 20 minutes. 6-Chloro-N-methyl-3-pyridinecarboxamide (93 mg, 0.55 mmol; may be prepared as described in Description 10 of WO2004056369) was added and the mixture heated at 90° C. under argon for 21 hours. The mixture was then cooled to room temperature, applied to an scx ion exchange column and eluted with methanol and then a solution of ammonia in methanol (2M). The basic fractions were evaporated under reduced pressure and the residue purified on the mass directed autoprep to afford the title compound (E9); MS (ES+) m/e 366 [M+H]+.

### EXAMPLE 10

### 6-{[2-(1-Pyrrolidinyl)-2,3-dihydro-1H-inden-5-yl]oxy}-3-pyridinecarboxamide (E10)

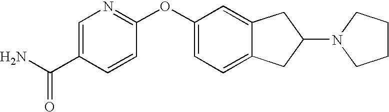

Sodium hydride (12 mg, 0.30 mmol, 60% in mineral oil) was added to a solution of 2-(1-pyrrolidinyl)-2,3-dihydro-1H-inden-5-ol (may be prepared as described in Description 4) (50 mg, 0.25 mmol) in dimethylformamide (3 ml) and the resulting mixture was stirred at room temperature for 20 minutes. 6-Chloro-3-pyridinecarboxamide (may be prepared as described in Description 10) (60 mg, 0.38 mmol) was added and the mixture heated at 90° C. under argon for 72 hours. The mixture was then cooled to room temperature, applied to an scx ion exchange column and eluted with methanol and then a solution of ammonia in methanol (2M). The basic fractions were evaporated under reduced pressure and the residue purified on the mass directed autoprep to afford the title compound (E10); MS (ES+) m/e 324 [M+H]+.

### EXAMPLES 11-14 (E11-E14)

The following examples were prepared from 2-(1-pyrrolidinyl)-2,3-dihydro-1H-inden-5-ol (may be prepared as described in Description 4) and the corresponding chloropyridine for the stated heating time using an analogous method to that described in Example 10:

| | | Heating | MS (ES+) m/e |
|---|---|---|---|
| Example | Chloropyridine | time | [M + H]⁺ |
| N-(1-Methylethyl)-6- | 6-Chloro-N-(1-methylethyl)- | 72 hours | 366 |
| {[2-(1-pyrrolidinyl)-2,3- | 3-pyridinecarboxamide | | |
| dihydro-1H-inden-5- | (may be prepared as | | |
| yl]oxy}-3- | described in Description 11) | | |
| pyridinecarboxamide | | | |
| (E11) | | | |
| 5-(1- | 2-Chloro-5-(1- | 72 hours | 378 |
| Pyrrolidinylcarbonyl)-2- | pyrrolidinylcarbonyl)pyridine | | |
| {[2-(1-pyrrolidinyl)-2,3- | (may be prepared as | | |
| dihydro-1H-inden-5- | described in Description 9) | | |
| yl]oxy}pyridine (E12) | | | |
| N-Ethyl-6-{[2-(1- | 6-Chloro-N-ethyl-3- | 18 hours | 352 |
| pyrrolidinyl)-2,3- | pyridinecarboxamide (may | | |
| dihydro-1H-inden-5- | be prepared as described | | |
| yl]oxy}-3- | in Description 12) | | |
| pyridinecarboxamide | | | |
| (E13) | | | |
| N,N-Dimethyl-6-{[2-(1- | 6-Chloro-N,N-dimethyl-3- | 18 hours | 352 |
| pyrrolidinyl)-2,3- | pyridinecarboxamide (may | | |
| dihydro-1H-inden-5- | be prepared as described | | |
| yl]oxy}-3- | in Description 13) | | |
| pyridinecarboxamide | | | |
| (E14) | | | |

### EXAMPLE 15

### N-Methyl-5-{[2-(1-pyrrolidinyl)-2,3-dihydro-1H-inden-5-yl]oxy}-2-pyrazinecarboxamide (E15)

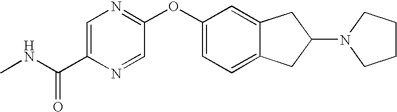

5-{[2-(1-Pyrrolidinyl)-2,3-dihydro-1H-inden-5-yl]oxy}-2-pyrazinecarboxylic acid (may be prepared as described in Example 7) (58 mg, 0.18 mmol) was dissolved in dichloromethane (3 ml), treated with N,N′-carbonyldiimidazole (58 mg, 0.36 mmol) and the resulting mixture heated at 40° C. under argon for 2 hours and then stirred at room temperature under argon for 72 hours. Methylamine (2M solution in tetrahydrofuran) (0.36 ml, 0.72 mmol) was added and the mixture stirred at room temperature for 2 hours. The mixture was diluted with methanol and applied to an scx ion exchange column and eluted with methanol and then a solution of ammonia in methanol (2M). The basic fractions were evaporated under reduced pressure and the residue purified on the mass directed autoprep to afford the title compound (E15); MS (ES+) m/e 339 [M+H]+.

### EXAMPLE 16

### 5-Bromo-2-({2-[(2S)-2-methyl-1-pyrrolidinyl]-2,3-dihydro-1H-inden-5-yl}oxy)pyridine (E16)

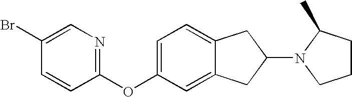

This compound was prepared from 2-[(2S)-2-Methyl-1-pyrrolidinyl]-2,3-dihydro-1H-inden-5-ol (may be prepared as described in Description 19) using an analogous method to that described in Example 2. MS (ES+) m/e 373 & 375 [M+H]⁺.

### EXAMPLE 17

### N-methyl-6-{[2-(1-piperidinyl)-2,3-dihydro-1H-inden-5-yl]oxy}-3-pyridine carboxamide (E17)

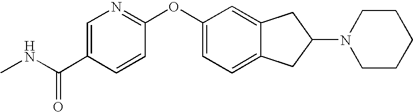

Sodium hydride (58 mg of a 60% dispersion in mineral oil, 1.4 mmol) was added to a solution of 2-(1-piperidinyl)-2,3-dihydro-1H-inden-5-ol (250 mg, 1.2 mmol; may be prepared as described in Description 15) in dimethylformamide (4 ml) and the mixture was stirred at room temperature for 20 minutes. 6-Chloro-N-methyl-3-pyridinecarboxamide (357 mg, 1.4 mmol; may be prepared as described in Description 10 of WO2004056369) was added and the mixture heated at 90° C. for 18 hours. The mixture was allowed to cool to room temperature and purified on a 10 g SCX ion exchange column and eluted with methanol and then a solution of ammonia in methanol (2M). The basic fractions were combined and evaporated. The residue was purified by column chromatography on silica eluting with 95-5 dichloromethane-2M ammonia in methanol solution. Fractions containing the product were combined and evaporated to give the title compound (E17). MS (ES+) m/e 352 [M+H]⁺.

### EXAMPLES 18-19 (E18-E19)

Examples 18 and 19 (E18 & E19) were prepared using an analogous method to that described in Example 17 from the appropriate starting material as shown in the table below:

| Product | Starting Material | MS Data |
|---|---|---|
| N-methyl-6-({2-[(2R)-2-methyl-1- | 2-[(2R)-2-Methyl-1- | MS (ES+) m/e |
| pyrrolidinyl]-2,3-dihydro-1H- | pyrrolidinyl]-2,3-dihydro-1H- | 352 [M + H]⁺. |
| inden-5-yl}oxy)-3- | inden-5-ol (may be prepared | |
| pyridinecarboxamide (E18) | as described in Description | |
| | 18) | |
| N-methyl-6-({2-[(2S)-2-methyl-1- | 2-[(2S)-2-Methyl-1- | MS (ES+) m/e |
| pyrrolidinyl]-2,3-dihydro-1H- | pyrrolidinyl]-2,3-dihydro-1H- | 352 [M + H]⁺. |
| inden-5-yl}oxy)-3- | inden-5-ol (may be prepared | |
| pyridinecarboxamide (E19) | as described in Description | |
| | 19) | |

### EXAMPLE 20

### 1-[6-({2-[(2S)-2-Methyl-1-pyrrolidinyl]-2,3-dihydro-1H-inden-5-yl}oxy)-3-pyridinyl]-2-pyrrolidinone (E20)

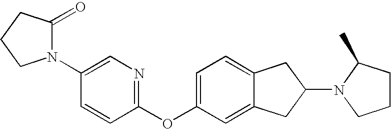

Example 20 was prepared using an analogous method to that described in Example 3 from 5-bromo-2-({2-[(2S)-2-methyl-1-pyrrolidinyl]-2,3-dihydro-1H-inden-5-yl}oxy)pyridine (may be prepared as described in Example 16). MS (ES+) m/e 378 [M+H]⁺.

### EXAMPLE 21

### (+)-Enantiomer N-methyl-6-{[2-(1-pyrrolidinyl)-2,3-dihydro-1H-inden-5-yl]oxy}-3-pyridinecarboxamide (E21)

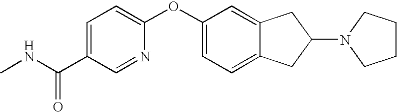

The racemic N-methyl-6-{[2-(1-pyrrolidinyl)-2,3-dihydro-1H-inden-5-yl]oxy}-3-pyridinecarboxamide (may be prepared as described in Example 1) was separated on a 250 mm×21.2 mm 10 micron particle size chiralcel OD column (pre-packed column purchased from Chiral Technologies) eluting with heptane:ethanol (90-10 v/v ratio; pump mixed) at a flow rate of 17 ml/min. Injection volume was 0.9 ml and detection was by U.V absorbance at 254 nm. The fraction containing the above enantiomer was evaporated under reduced pressure to obtain the title compound (E21) which had an [α]_{D}=+84.4° (MeOH) MS (ES+) m/e 338 [M+H]⁺.

### EXAMPLE 22

### (−)-Enantiomer N-methyl-6-{[2-(1-pyrrolidinyl)-2,3-dihydro-1H-inden-5-yl]oxy}-3-pyridinecarboxamide (E22)

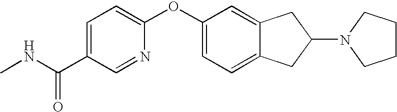

The racemic N-methyl-6-{[2-(1-pyrrolidinyl)-2,3-dihydro-1H-inden-5-yl]oxy}-3-pyridinecarboxamide (may be prepared as described in Example 1) was separated on a 250 mm×21.2 mm 10 micron particle size chiralcel OD column (pre-packed column purchased from Chiral Technologies) eluting with heptane:ethanol (90-10 v/v ratio; pump mixed) at a flow rate of 17 ml/min. Injection volume was 0.9 ml and detection was by U.V absorbance at 254 nm. The fraction containing the above enantiomer was evaporated under reduced pressure to obtain the title compound (E22) which had an [α]_{D}=−50.0° (MeOH) MS (ES+) m/e 338 [M+H]⁺.

### EXAMPLE 23

### 5-Iodo-2-{[2-(1-pyrrolidinyl)-2,3-dihydro-1H-inden-5-yl]oxy}pyridine

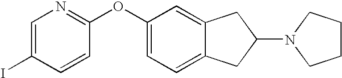

A mixture of 2-(1-pyrrolidinyl)-2,3-dihydro-1H-inden-5-ol (may be prepared as described in Description 4) (406 mg, 2 mmol), potassium carbonate (828 mg, 6 mmol) and 2-chloro-5-iodopyridine (574 mg, 2.4 mmol) in dry dimethylformamide (5 ml) was heated at 150° C. in an Emrys Optimiser microwave for 7 hours. Potassium carbonate (414 mg, 3 mmol) was added and heating at 150° C. in an Emrys Optimiser microwave continued for a further 4 hours. The mixture was filtered through Celite and the filtrate evaporated. The residue was purified on a 10 g SCX ion exchange cartridge, eluting with methanol then 2M ammonia in methanol. The basic fractions were evaporated and the residue further purified by flash chromatography on silica gel eluting with a mixture of 2M ammonia in methanol and dichloromethane (1:99-5:95) to afford the title compound (E23) as a light brown solid (248 mg, 31%); NMR (CDCl3) δ 8.35 (1H, d), 7.89 (1H, d), 7.18 (1H, d), 6.93 (1H, d) 6.87 (1H, dd), 3.14-2.89 (5H, m), 2.62 (4H, m), 1.88-1.82 (4H, m).

### EXAMPLE 24

### 1-{5-[(4-Bromophenyl)oxy]-2,3-dihydro-1H-inden-2-yl}pyrrolidine (E24)

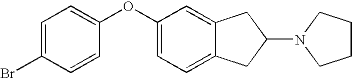

A mixture of 2-(1-pyrrolidinyl)-2,3-dihydro-1H-inden-5-ol (may be prepared as described in Description 4) (87 mg, 0.43 mmol), 4-bromophenylboronic acid (86 mg, 0.43 mmol), copper(II) acetate (116 mg, 0.64 mmol), triethylamine (300 ul, 2.14 mmol) and powdered 4 A molecular sieve (200 mg) was stirred in dichloromethane (3 ml) at room temperature for 17 hours. 4-Bromophenylboronic acid (17 mg, 0.09 mmol) was added and stirring continued for a further 72 hours. Dichloromethane was added and the mixture filtered through Celite and the filtrate evaporated. The residue was purified on a 5 g SCX ion exchange cartridge, eluting with methanol then 2M ammonia in methanol. The basic fractions were evaporated and the residue further purified by flash chromatography on silica gel eluting with a mixture of 2M ammonia in methanol and dichloromethane (4:96) to afford the title compound (E24). (45 mg, 29%) MS m/e 360, 361 [M+H]⁺.

### EXAMPLE 25

### 1-(4-{[2-(1-Pyrrolidinyl)-2,3-dihydro-1H-inden-5-yl]oxy}phenyl)-2-pyrrolidinone (E25)

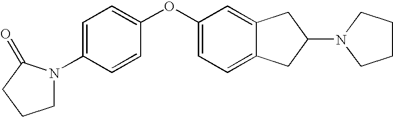

1-{5-[(4-Bromophenyl)oxy]-2,3-dihydro-1H-inden-2-yl}pyrrolidine (45 mg, 0.126 mmol; may be prepared as described in Example 24), 2-pyrrolidinone (22 mg, 0.252 mmol), copper(I) iodide (3 mg, 0.013 mmol), N,N′-dimethylethylenediamine (1.5 mg, 0.013 mmol) and potassium carbonate (63 mg, 0.452 mmol) were suspended in 1,4-dioxan (2 ml) and heated at 150° C. in an Emrys Optimiser microwave for 24 hours. The mixture was filtered through Celite and the filtrate evaporated. The residue was purified by flash chromatography eluting with a mixture of 2M ammonia in methanol and dichloromethane (4:96). The product was dissolved in dichloromethane and 1M hydrogen chloride in diethyl ether (1 ml) added and the mixture stirred for 5 minutes. The solvent was evaporated and the residue treated with diethyl ether, stirred for 5 minutes and the solvent decanted. The solid was dried in vacuo to afford the title compound (E25). (5 mg, 11%) MS m/e 363 [M+H]⁺.

### EXAMPLE 26

### 1Methyl-3-(6-{[2-(1-pyrrolidinyl)-2,3-dihydro-1H-inden-5-yl]oxy}-3-pyridinyl)-2-imidazolidinone (E26)

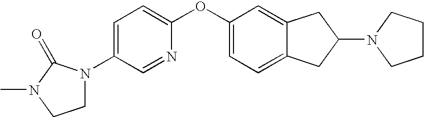

5-Iodo-2-{[2-(1-pyrrolidinyl)-2,3-dihydro-1H-inden-5-yl]oxy}pyridine (100 mg, 0.25 mmol; may be prepared as described in Example 23), 1-methyl-2-imidazolidinone (50 mg, 0.5 mmol), copper(I) iodide (5 mg, 0.025 mmol), N,N′-dimethylethylenediamine (3 mg, 0.025 mmol) and potassium carbonate (122 mg, 0.88 mmol) were suspended in 1,4-dioxan (4 ml) and heated at 150° C. in an Emrys Optimiser microwave for 5 hours. The mixture was filtered through Celite and the filtrate evaporated. The residue was purified by flash chromatography eluting with a mixture of 2M ammonia in methanol and dichloromethane (4:96). The product was dissolved in dichloromethane and 1M hydrogen chloride in diethyl ether (1 ml) added and the mixture stirred for 5 minutes. The solvent was evaporated and the residue treated with diethyl ether, stirred for 5 minutes and the solvent decanted. The solid was dried in vacuo to afford the title compound (E26). (34 mg, 36%) MS m/e 379 [M+H]⁺.

### EXAMPLE 27

### 3-(6-{[2-(1-Pyrrolidinyl)-2,3-dihydro-1H-inden-5-yl]oxy}-3-pyridinyl)-1,3-oxazolidin-2-one (E27)

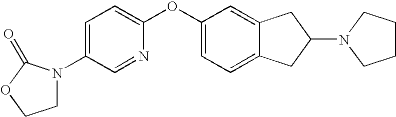

5-Iodo-2-{[2-(1-pyrrolidinyl)-2,3-dihydro-1H-inden-5-yl]oxy}pyridine (100 mg, 0.25 mmol; may be prepared as described in Example 23), 2-oxazolidone (44 mg, 0.5 mmol), copper(I) iodide (5 mg, 0.025 mmol), N,N′-dimethylethylenediamine (3 mg, 0.025 mmol) and potassium carbonate (122 mg, 0.88 mmol) were suspended in 1,4-dioxan (4 ml) and heated at 150° C. in an Emrys Optimiser microwave for 8 hours. The mixture was filtered through Celite and the filtrate evaporated. The residue was purified twice by flash chromatography eluting with a mixture of 2M ammonia in methanol and dichloromethane (4:96). The product was dissolved in dichloromethane and 1M hydrogen chloride in diethyl ether (1 ml) added and the mixture stirred for 5 minutes. The solvent was evaporated and the residue treated with diethyl ether, stirred for 5 minutes and the solvent decanted. The solid was dried in vacuo to afford the title compound (E27). (9 mg, 10%) MS m/e 366 [M+H]⁺.

All publications, including but not limited to patents and patent applications, cited in this specification are herein incorporated by reference as if each individual publication were specifically and individually indicated to be incorporated by reference herein as though fully set forth.

### Biological Data

A membrane preparation containing histamine H3 receptors may be prepared in accordance with the following procedures:

### (i) Generation of Histamine H3 Cell Line

DNA encoding the human histamine H3 gene (Huvar, A. et al. (1999) Mol. Pharmacol. 55(6), 1101-1107) was cloned into a holding vector, pcDNA3.1 TOPO (InVitrogen) and its cDNA was isolated from this vector by restriction digestion of plasmid DNA with the enzymes BamH1 and Not-1 and ligated into the inducible expression vector pGene (InVitrogen) digested with the same enzymes. The GeneSwitch™ system (a system where in transgene expression is switched off in the absence of an inducer and switched on in the presence of an inducer) was performed as described in U.S. Pat. Nos. 5,364,791; 5,874,534; and 5,935,934. Ligated DNA was transformed into competent DH5α E. coli host bacterial cells and plated onto Luria Broth (LB) agar containing Zeocin™ (an antibiotic which allows the selection of cells expressing the sh ble gene which is present on pGene and pSwitch) at 50 μg ml⁻¹. Colonies containing the re-ligated plasmid were identified by restriction analysis. DNA for transfection into mammalian cells was prepared from 250 ml cultures of the host bacterium containing the pGeneH3 plasmid and isolated using a DNA preparation kit (Qiagen Midi-Prep) as per manufacturers guidelines (Qiagen).

CHO K1 cells previously transfected with the pSwitch regulatory plasmid (InVitrogen) were seeded at 2×10e6 cells per T75 flask in Complete Medium, containing Hams F12 (GIBCOBRL, Life Technologies) medium supplemented with 10% v/v dialysed foetal bovine serum, L-glutamine, and hygromycin (100 μg ml⁻¹), 24 hours prior to use. Plasmid DNA was transfected into the cells using Lipofectamine plus according to the manufacturers guidelines (InVitrogen). 48 hours post transfection cells were placed into complete medium supplemented with 500 μg ml⁻¹ Zeocin™.

10-14 days post selection 10 nM Mifepristone (InVitrogen), was added to the culture medium to induce the expression of the receptor. 18 hours post induction cells were detached from the flask using ethylenediamine tetra-acetic acid (EDTA; 1:5000; InVitrogen), following several washes with phosphate buffered saline pH 7.4 and resuspended in Sorting Medium containing Minimum Essential Medium (MEM), without phenol red, and supplemented with Earles salts and 3% Foetal Clone II (Hyclone). Approximately 1×10e7 cells were examined for receptor expression by staining with a rabbit polyclonal antibody, 4a, raised against the N-terminal domain of the histamine H3 receptor, incubated on ice for 60 minutes, followed by two washes in sorting medium. Receptor bound antibody was detected by incubation of the cells for 60 minutes on ice with a goat anti rabbit antibody, conjugated with Alexa 488 fluorescence marker (Molecular Probes). Following two further washes with Sorting Medium, cells were filtered through a 50 μm Filcon™ (BD Biosciences) and then analysed on a FACS Vantage SE Flow Cytometer fitted with an Automatic Cell Deposition Unit. Control cells were non-induced cells treated in a similar manner. Positively stained cells were sorted as single cells into 96-well plates, containing Complete Medium containing 500 μg ml⁻¹ Zeocin™ and allowed to expand before reanalysis for receptor expression via antibody and ligand binding studies. One clone, 3H3, was selected for membrane preparation.

(ii) Membrane Preparation from Cultured Cells

All steps of the protocol are carried out at 4° C. and with pre-cooled reagents. The cell pellet is resuspended in 10 volumes of buffer A2 containing 50 mM N-2-hydroxyethylpiperazine-N′-2-ethanesulfonic acid (HEPES) (pH 7.40) supplemented with 10e-4M leupeptin (acetyl-leucyl-leucyl-arginal; Sigma L2884), 25 μg/ml bacitracin (Sigma B0125), 1 mM ethylenediamine tetra-acetic acid (EDTA), 1 mM phenylmethylsulfonyl fluoride (PMSF) and 2×10e−6M pepstain A (Sigma). The cells are then homogenised by 2×15 second bursts in a 1 litre glass Waring blender, followed by centrifugation at 500 g for 20 minutes. The supernatant is then spun at 48,000 g for 30 minutes. The pellet is resuspended in 4 volumes of buffer A2 by vortexing for 5 seconds, followed by homogenisation in a Dounce homogeniser (10-15 strokes). At this point the preparation is aliquoted into polypropylene tubes and stored at −70° C.

(iii) Generation of Histamine H1 Cell Line

The human H1 receptor was cloned using known procedures described in the literature [Biochem. Biophys. Res. Commun. 1994, 201(2), 894]. Chinese hamster ovary cells stably expressing the human H1 receptor were generated according to known procedures described in the literature [Br. J. Pharmacol. 1996, 117(6), 1071].

Compounds of the invention may be tested for in vitro biological activity in accordance with the following assays:

### (II) Histamine H3 Functional Antagonist Assay

For each compound being assayed, in a solid white 384 well plate, is added:—

(a) 0.5 μl of test compound diluted to the required concentration in DMSO (or 0.5 μl DMSO as a control);
(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N2-Hydroxyethylpiperazine-N′-2-ethanesulfonic acid (HEPES)+100 mM NaCl+10 mM MgCl₂, pH7.4 NaOH) to give a final volume of 30 μl which contains 5 μg protein and 0.25 mg bead per well, incubating at room temperature for 60 minutes on a roller and, just prior to addition to the plate, adding 10 μM final concentration of guanosine 5′ diphosphate (GDP)(Sigma; diluted in assay buffer);
(c) 15 μl 0.38 nM [³⁵S]-GTPγS (Amersham; Radioactivity concentration=37 MBq/ml; Specific activity=1160 Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

### (III) Histamine H1 Functional Antagonist Assay

Compounds are assayed in a black walled clear bottom 384-well plate with cells seeded at 10000 cells/well. Tyrodes buffer is used throughout (NaCl 145 mM, KCl 2.5 mM, HEPES 10 mM, glucose 1 mM, MgCl₂ 1.2 mM, CaCl₂1.5 mM, probenecid 2.5 mM, pH adjusted to 7.40 with NaOH 1.0 M). Each well is treated with 10 μl of a solution of FLUO4AM (10 μM in Tyrodes buffer at pH 7.40) and plates are then incubated for 60 minutes at 37° C. Wells are then washed with Tyrodes buffer using a EMBLA cell washer system, leaving 40 μl buffer in each well, and then treated with 10 μl of test compound in Tyrodes buffer. Each plate is incubated for 30 min to allow equilibration of the test compound with the receptor. Each well is then treated with 10 μl of histamine solution in Tyrodes buffer.

Functional antagonism is indicated by a suppression of histamine induced increase in fluorescence, as measured by the FLIPR system (Molecular Devices). By means of concentration effect curves, functional potencies are determined using standard pharmacological mathematical analysis.

### Results

Hydrochloride salts of the compounds of Examples E1, E3-E5, E8-E15, E17-E22 and E25-E27 were tested in the histamine H3 functional antagonist assay. The results are expressed as functional pKᵢ (fpKᵢ) values. A functional pKi is the negative logarithm of the antagonist equilibrium dissociation constant as determined in the H3 functional antagonist assay using membrane prepared from cultured H3 cells. The results given are averages of a number of experiments. The salts exhibited antagonism >7.5 fpKᵢ. More particularly, the hydrochloride salts of the compounds of Examples 3, 4, 12, 20 and 22 exhibited antagonism >9.0 fpKᵢ.

Hydrochloride salts of the compounds of Examples E1, E3-E5, E8-E15, E17-E22 and E25-E27 were tested in the histamine H1 functional antagonist assay. The results are expressed as functional pKᵢ (fpKᵢ) values and are averages of a number of experiments. The functional pKi may be derived from the negative logarithm of the plC50 (concentration producing 50% inhibition) in the H1 functional antagonist assay according to the Cheng-Prusoff equation (Cheng, Y. C. and Prusoff, W. H., 1973, Biochem. Pharmacol. 22, 3099-3108.). All compounds tested exhibited antagonism <6.0 fpKᵢ.

What is claimed is:

1.-15. (canceled)

16. A compound of formula (I) or a pharmaceutically acceptable salt thereof: 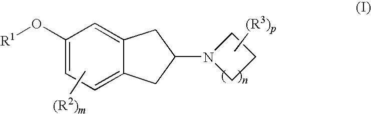
wherein:
R¹ represents —X—C₃₋₈ cycloalkyl, —X-aryl, —X-heterocyclyl, —X-heteroaryl, —X—C₃₋₈ cycloalkyl-Y—C₃₋₈ cycloalkyl, —X—C₃₋₈ cycloalkyl-Y-aryl, —X—C₃₋₈ cycloalkyl-Y-heteroaryl, —X—C₃₋₈ cycloalkyl-Y-heterocyclyl, —X-aryl-Y—C₃₋₈ cycloalkyl, —X-aryl-Y-aryl, —X-aryl-Y-heteroaryl, —X-aryl-Y-heterocyclyl, —X-heteroaryl-Y—C₃₋₈ cycloalkyl, —X-heteroaryl-Y-aryl, —X-heteroaryl-Y-heteroaryl, —X-heteroaryl-Y-heterocyclyl, —X-heterocyclyl-Y—C₃₋₈ cycloalkyl, —X-heterocyclyl-Y-aryl, —X-heterocyclyl-Y-heteroaryl, or —X-heterocyclyl-Y-heterocyclyl;
X represents a bond or C₁₋₆ alkyl;
Y represents a bond, C₁₋₆ alkyl, CO, CONH, COC₂₋₆ alkenyl, O, SO₂ or NHCOC₁₋₆ alkyl;
R² represents halogen, C₁₋₆ alkyl, C₁₋₆ alkoxy, cyano, or amino;
m represents an integer from 0 to 2;
n represents an integer from 1 to 4;
R³ represents hydrogen, fluorine, or —C₁₋₃ alkyl;
p represents an integer from 0 to 2;
wherein said alkyl, cycloalkyl, aryl, heteroaryl and heterocyclyl groups of R¹ may be optionally substituted by one or more substituents which may be the same or different, and which are selected from the group consisting of halogen, hydroxy, cyano, nitro, ═O, haloC₁₋₆ alkyl, haloC₁₋₆ alkoxy, C₁₋₆ alkyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₅ alkoxy, C₁₋₆ alkanoyl, C₁₋₆ alkoxycarbonyl, C₁₋₆ alkylsulfonyl, C₁₋₆ alkylsulfonyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁴, —CO₂R⁴, —COR⁴, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵COR⁶, —NR⁴CONR⁵R⁶ or —SO₂NR⁵R⁶, wherein R⁴, R⁵ and R⁶ independently represent hydrogen, C₁₋₆ alkyl, —C₃₋₈ cycloalkyl, —C₁₋₆ alkyl-C₃₋₈ cycloalkyl, aryl, heterocyclyl or heteroaryl or —NR⁵R⁶ may represent a nitrogen containing heterocyclyl group, wherein said R⁴, R⁵ and R⁶ groups may be optionally substituted by one or more substituents which may be the same or different, and which are selected from the group consisting of halogen, hydroxy, C₁₋₆ alkyl, C₁₋₆ alkoxy, cyano, amino, ═O or haloC₁₋₆ alkyl, provided that where R¹ represents —C₃₋₆ alkyl, R¹ is not substituted by hydroxy, C₁₋₆ alkoxycarbonyl or —CO₂R⁴;
or solvates thereof.

17. A compound according to claim 16, wherein R¹ represents —X-aryl, —X-aryl-Y-heterocyclyl, —X-heterocyclyl-Y-heterocyclyl, —X-heteroaryl or —X-heteroaryl-Y-heterocyclyl.

18. A compound according to claim 17, wherein R¹ represents:
—X-heteroaryl optionally substituted by a —CONR⁶R⁷ group, a —CO₂R⁴ group or a halogen atom; or
—X-heteroaryl-Y-heterocyclyl optionally substituted on the heterocyclic group by an oxo group and/or an —R⁴ group.

19. A compound according to claim 16, wherein X represents a bond.

20. A compound according to claim 16, wherein Y represents a bond or CO.

21. A compound according to claim 16, wherein m represents 0.

22. A compound according to claim 16, wherein n represents 2.

23. A compound according to claim 16, wherein p represents 0.

24. A compound according to claim 16 which is:
N-Methyl-6-{[2-(1-pyrrolidinyl)-2,3-dihydro-1H-inden-5-yl]oxy}-3-pyridinecarboxamide;
5-Bromo-2-{[2-(1-pyrrolidinyl)-2,3-dihydro-1H-inden-5-yl]oxy}pyridine;
1-(6-{[2-(1-Pyrrolidinyl)-2,3-dihydro-1H-inden-5-yl]oxy}-3-pyridinyl)-2-pyrrolidinone inclusive of its (+) and (−) enantiomers;
Methyl 5-{[2-(1-pyrrolidinyl)-2,3-dihydro-1H-inden-5-yl]oxy}-2-pyrazinecarboxylate;
5-{[2-(1-Pyrrolidinyl)-2,3-dihydro-1H-inden-5-yl]oxy}-2-pyrazinecarboxylic acid;
6-{[2-(Hexahydro-1H-azepin-1-yl)-2,3-dihydro-1H-inden-5-yl]oxy}-N-methyl-3-pyridinecarboxamide;
6-({2-[(2R,5R)-2,5-dimethyl-1-pyrrolidinyl]-2,3-dihydro-1H-inden-5-yl}oxy)-N-methyl-3-pyridinecarboxamide;
6-{[2-(1-Pyrrolidinyl)-2,3-dihydro-1H-inden-5-yl]oxy}-3-pyridinecarboxamide;
N-(1-Methylethyl)-6-{[2-(1-pyrrolidinyl)-2,3-dihydro-1H-inden-5-yl]oxy}-3-pyridinecarboxamide;
5-(1-Pyrrolidinylcarbonyl)-2-{[2-(1-pyrrolidinyl)-2,3-dihydro-1H-inden-5-yl]oxy}pyridine;
N-Ethyl-6-{[2-(1-pyrrolidinyl)-2,3-dihydro-1H-inden-5-yl]oxy}-3-pyridinecarboxamide;
N,N-Dimethyl-6-{[2-(1-pyrrolidinyl)-2,3-dihydro-1H-inden-5-yl]oxy}-3-pyridinecarboxamide;
N-Methyl-5-{[2-(1-pyrrolidinyl)-2,3-dihydro-1H-inden-5-yl]oxy}-2-pyrazinecarboxamide;
5-Bromo-2-({2-[(2S)-2-methyl-1-pyrrolidinyl]-2,3-dihydro-1H-inden-5-yl}oxy)pyridine;
N-methyl-6-{[2-(1-piperidinyl)-2,3-dihydro-1H-inden-5-yl]oxy}-3-pyridine carboxamide;
N-methyl-6-({2-[(2R)-2-methyl-1-pyrrolidinyl]-2,3-dihydro-1H-inden-5-yl}oxy)-3-pyridinecarboxamide;
N-methyl-6-({2-[(2S)-2-methyl-1-pyrrolidinyl]-2,3-dihydro-1H-inden-5-yl}oxy)-3-pyridinecarboxamide;
1-[6-({2-[(2S)-2-Methyl-1-pyrrolidinyl]-2,3-dihydro-1H-inden-5-yl}oxy)-3-pyridinyl]-2-pyrrolidinone;
(+)-Enantiomer N-methyl-6-{[2-(1-pyrrolidinyl)-2,3-dihydro-1H-inden-5-yl]oxy}-3-pyridinecarboxamide;
(−)-Enantiomer N-methyl-6-{[2-(1-pyrrolidinyl)-2,3-dihydro-1H-inden-5-yl]oxy}-3-pyridinecarboxamide;
5-Iodo-2-{[2-(1-pyrrolidinyl)-2,3-dihydro-1H-inden-5-yl]oxy}pyridine;
1-{5-[(4-Bromophenyl)oxy]-2,3-dihydro-1H-inden-2-yl}pyrrolidine;
1-(4-{[2-(1-Pyrrolidinyl)-2,3-dihydro-1H-inden-5-yl]oxy}phenyl)-2-pyrrolidinone;
1-Methyl-3-(6-{[2-(1-pyrrolidinyl)-2,3-dihydro-1H-inden-5-yl]oxy}-3-pyridinyl)-2-imidazolidinone;
3-(6-{[2-(1-Pyrrolidinyl)-2,3-dihydro-1H-inden-5-yl]oxy}-3-pyridinyl)-1,3-oxazolidin-2-one;
or a pharmaceutically acceptable salt or solvate thereof.

25. A pharmaceutical composition which comprises the compound of formula (I) as defined in claim 16 or a pharmaceutically acceptable salt or solvate thereof and a pharmaceutically acceptable carrier or excipient.

26. A method of treatment of neurological diseases which comprises administering to a host in need thereof an effective amount of a compound of formula (I) as defined in claim 16 or a pharmaceutically acceptable salt or solvate thereof.
